(19) United States
(12) Patent Application Publication
(10) Pub. No.: US 2018/0301492 A1
(43) Pub. Date: Oct. 18, 2018

(54) SOLID-STATE IMAGING DEVICE

(71) Applicant: OLYMPUS CORPORATION, Tokyo (JP)

(72) Inventor: Yoshiaki Takemoto, Tokyo (JP)

(73) Assignee: OLYMPUS CORPORATION, Tokyo (JP)

(21) Appl. No.: 16/006,147

(22) Filed: Jun. 12, 2018

Related U.S. Application Data
(63) Continuation of application No. PCT/JP2015/085355, filed on Dec. 17, 2015.

(51) Int. Cl. H01L 27/146; H01L 27/32; A61B 5/00

(52) U.S. Cl. CPC H01L 27/14623; H01L 27/3234; H01L 27/14645; H01L 27/1462; A61B 2562/046; H01L 27/14627; H01L 27/14636; A61B 5/0071; A61B 2562/0233; H01L 27/14621

(57) ABSTRACT

A solid-state imaging device includes a first layer, a second layer, a plurality of first microlenses, and a plurality of second microlenses. A first light blocking film of the second layer is arranged in a region corresponding to the plurality of photoelectric conversion elements of the first layer. The plurality of first microlenses are arranged in a region, which corresponds to the plurality of photoelectric conversion elements, on the first principal surface of the first layer. The plurality of second microlenses are arranged in a region which is different from a region corresponding to the first light blocking film in a fourth principal surface of the second layer and which corresponds to a first light transmission layer of the first layer.

The present application is a continuous application based on PCT/JP2015/085355 filed on Dec. 17, 2015, the content of which is incorporated herein by reference.

### BACKGROUND OF THE INVENTION

### Field of the Invention

The present invention relates to a solid-state imaging device.

### Description of Related Art

In general, video cameras, electronic still cameras and the like have been widely used. As these cameras, charge coupled device (CCD) type and amplification type solid-state imaging devices are used. In an amplification type solid-state imaging device, signal charge generated and accumulated in photoelectric conversion elements of pixels receiving light is transmitted to amplification units provided in the pixels. An amplification type solid-state imaging device outputs signals amplified by the amplification units from the pixels. In the amplification type solid-state imaging device, a plurality of pixels configured as above are arranged in a matrix form. A complementary metal oxide semiconductor (CMOS) type solid-state imaging device, which uses a CMOS transistor, and the like are an example of an amplification type solid-state imaging device.

For example, when a solid-state imaging device is used as a digital scanner, images are captured in a state in which the solid-state imaging device is in close contact with an object to be captured, in many cases. A light guide is provided on a surface of a sensor of such a contact type solid-state imaging device. The light guide transmits light reflected by an object. The sensor detects the light transmitted by the light guide. In the vicinity of the contact type solid-state imaging device, a light source needs to be provided. In the contact type solid-state imaging device, a line sensor in which photoelectric conversion elements are one-dimensionally arranged is generally used. In such a configuration, the arrangement of the light guide and the light source is restricted and miniaturization of a digital scanner is restricted. In order to scan two-dimensionally by using the line sensor, it is necessary to mechanically drive the solid-state imaging device.

In consideration of the aforementioned situation, a contact type solid-state imaging device, in which the restriction of arrangement is reduced, has been disclosed. For example, in the technology disclosed in Japanese Unexamined Patent Application, First Publication No. H6-260627, a plurality of microlenses are arranged on a surface of a substrate facing an object. Light incident on a rear surface of the substrate passes through the substrate and is irradiated onto the object. Light reflected by the object passes through the plurality of microlenses and is incident on a sensor in a silicon thin film, that is, a photoelectric conversion element. Due to this configuration, improvement of resolution is expected. On the rear surface side of the substrate, a light blocking film is arranged on a position facing the photoelectric conversion element. Some of the light incident on the rear surface of the substrate is blocked by the light blocking film.

### SUMMARY OF THE INVENTION

According to the first aspect of the present invention, a solid-state imaging device includes a first layer, a second layer, a plurality of first microlenses, and a plurality of second microlenses. The first layer includes a first principal surface, a second principal surface, a plurality of photoelectric conversion elements, and a first light transmission layer. The first principal surface and the second principal surface face opposite directions. The plurality of photoelectric conversion elements receives light incident on the first principal surface. The first light transmission layer allows light, which is incident on a second region of the second principal surface different from a first region of the second principal surface corresponding to the plurality of photoelectric conversion elements, to be emitted from the first principal surface. The second layer includes a third principal surface, a fourth principal surface, a first light blocking film, and a second light transmission layer. The third principal surface and the fourth principal surface face opposite directions. The third principal surface faces the second principal surface. The first light blocking film is arranged in a third region of the second layer corresponding to the plurality of photoelectric conversion elements and blocks light incident on the fourth principal surface. The second light transmission layer allows light, which is incident on a fifth region of the fourth principal surface that is different from a fourth region of the fourth principal surface corresponding to the first light blocking film and that corresponds to the first light transmission layer, to be emitted from the third principal surface. The plurality of first microlenses are arranged in a sixth region of the first principal surface which corresponds to the plurality of photoelectric conversion elements and have a convex shape toward an outer side of the first principal surface. The plurality of second microlenses are arranged in the fifth region and have a convex shape toward an outer side of the fourth principal surface.

According to the second aspect of the present invention, in the first aspect, the solid-state imaging device may further include a third layer and a support substrate. The third layer may include the plurality of second microlenses. The third layer may face the fourth principal surface. The third layer may be arranged between the second layer and the support substrate and may allow light incident on the third layer to pass therethrough. The support substrate may include a fifth principal surface and a sixth principal surface and allow light incident on the support substrate to pass therethrough. The fifth principal surface and the sixth principal surface may face opposite directions. The fifth principal surface may face the third layer.

According to the third aspect of the present invention, in the first aspect, the plurality of first microlenses may be arranged only in the sixth region. The plurality of second microlenses may be arranged only in the fifth region.

According to the fourth aspect of the present invention, in the first aspect, the plurality of first microlenses may be arranged in the sixth region and a seventh region of the first principal surface which corresponds to the first light transmission layer.

According to the fifth aspect of the present invention, in the first aspect, the plurality of second microlenses may be arranged in the fifth region and the fourth region.

According to the sixth aspect of the present invention, in the first aspect, the solid-state imaging device may further include a plurality of transistors. The plurality of transistors may be electrically connected to the plurality of photoelectric conversion elements and may be arranged between the plurality of photoelectric conversion elements and the fourth principal surface.

According to the seventh aspect of the present invention, in the first aspect, the second layer may further include a plurality of wirings including a metal.

According to the eighth aspect of the present invention, in the first aspect, a focal distance of each of the plurality of first microlenses may be shorter than a focal distance of each of the plurality of second microlenses.

According to the ninth aspect of the present invention, in the first aspect, a width of the first light transmission layer arranged between two adjacent photoelectric conversion elements may be larger than an interval between the first light blocking films respectively corresponding to the two adjacent photoelectric conversion elements.

According to the tenth aspect of the present invention, in the first aspect, the first light transmission layer may be arranged in a groove formed between the plurality of photoelectric conversion elements.

According to the eleventh aspect of the present invention, in the first aspect, the first layer may further include a second light blocking film. The second light blocking film may be arranged on a side surface of the groove and may block light incident on the first light transmission layer.

According to the twelfth aspect of the present invention, in the first aspect, the solid-state imaging device may further include a plurality of color filters. The plurality of color filters may be arranged in the sixth region and may be arranged between the plurality of photoelectric conversion elements and the plurality of first microlenses.

According to the thirteenth aspect of the present invention, in the first aspect, the solid-state imaging device may further include a third layer and a filter. The third layer may include the plurality of second microlenses. The third layer may face the fourth principal surface. The third layer may be arranged between the second layer and the filter. The third layer may allow light incident on the third layer to pass therethrough. The filter may have a structure in which a plurality of films including a dielectric are stacked.

According to the fourteenth aspect of the present invention, in the first aspect, the solid-state imaging device may further include a filter. The filter may have a structure in which a plurality of films including a dielectric are stacked and may be arranged between the second layer and the plurality of second microlenses.

According to the fifteenth aspect of the present invention, in the first aspect, the first light blocking film may include a conductive material and a power supply voltage or a ground voltage may be applied to the first light blocking film.

According to the sixteenth aspect of the present invention, in the second aspect, the solid-state imaging device may further include a light emitting element. The light emitting element may include a first electrode layer, a second electrode layer, and a light emitting layer. The first electrode layer, the second electrode layer, and the light emitting layer may be stacked in a thickness direction of the support substrate. The first electrode layer may face the sixth principal surface. The light emitting layer may be arranged between the first electrode layer and the second electrode layer.

According to the seventeenth aspect of the present invention, in the first aspect, the solid-state imaging device may further include a third layer, a support substrate, and a light emitting element. The third layer may include the plurality of second microlenses. The third layer may face the fourth principal surface. The third layer may be arranged between the second layer and the light emitting element. The third layer may allow light incident on the third layer to pass therethrough. The support substrate may include a fifth principal surface and a sixth principal surface. The fifth principal surface and the sixth principal surface may face opposite directions. The light emitting element may include a first electrode layer, a second electrode layer, and a light emitting layer. The first electrode layer, the second electrode layer, and the light emitting layer may be stacked in a thickness direction of the support substrate. The first electrode layer may face the third layer. The second electrode layer may face the fifth principal surface. The light emitting layer may be arranged between the first electrode layer and the second electrode layer.

According to the eighteenth aspect of the present invention, in the sixteenth aspect, the light emitting layer may include an organic light emitting material.

According to the nineteenth aspect of the present invention, in the seventeenth aspect, the light emitting layer may include an organic light emitting material.

### BRIEF DESCRIPTION OF THE DRAWINGS

FIG. 1 is a sectional view of a solid-state imaging device according to a first embodiment of the present invention.

FIG. 2 is a plan view of the solid-state imaging device according to the first embodiment of the present invention.

FIG. 3 is a plan view of the solid-state imaging device according to the first embodiment of the present invention.

FIG. 4 is a sectional view of a solid-state imaging device according to a second embodiment of the present invention.

FIG. 5 is a sectional view of a solid-state imaging device according to a third embodiment of the present invention.

FIG. 6 is a sectional view of a solid-state imaging device according to a fourth embodiment of the present invention.

FIG. 7 is a sectional view of a solid-state imaging device according to a fifth embodiment of the present invention.

FIG. 8 is a plan view of the solid-state imaging device according to the fifth embodiment of the present invention.

FIG. 9 is a plan view of the solid-state imaging device according to the fifth embodiment of the present invention.

FIG. 10 is a sectional view of a solid-state imaging device according to a sixth embodiment of the present invention.

FIG. 11 is a sectional view of a solid-state imaging device according to a modification example of the sixth embodiment of the present invention.

FIG. 12 is a sectional view of a solid-state imaging device according to a seventh embodiment of the present invention.

FIG. 13 is a sectional view of a solid-state imaging device according to a modification example of the seventh embodiment of the present invention.

### DETAILED DESCRIPTION OF THE INVENTION

With reference to the drawings, embodiments of the present invention will be described. In each of the following embodiments, an example of a contact type solid-state imaging device will be described. The solid-state imaging device of each embodiment need not be always used in a state of contacting with an object.

### First Embodiment

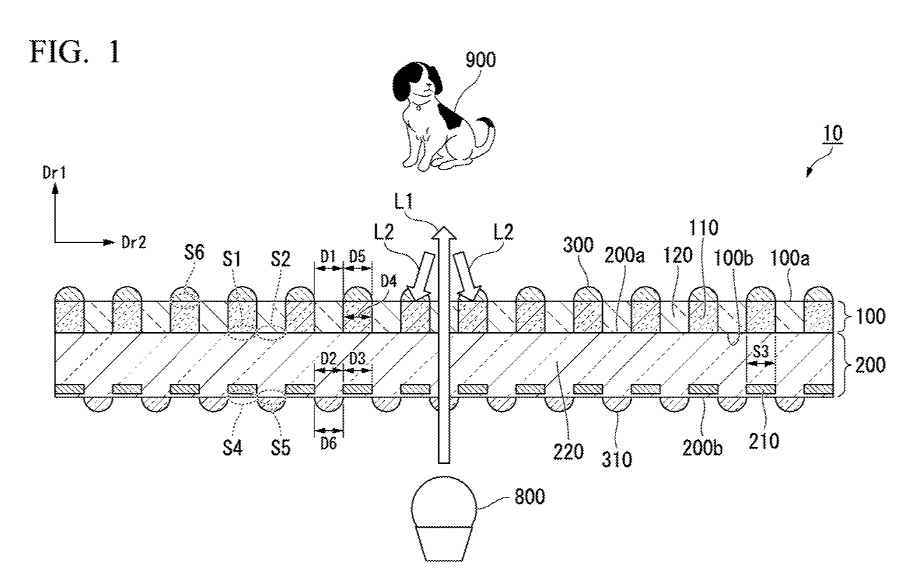

FIG. 1 shows a configuration of a solid-state imaging device 10 according to a first embodiment of the present invention. FIG. 1 shows a section of the solid-state imaging device 10. Dimensions of parts constituting the solid-state imaging device 10 are not limited to the dimension shown in FIG. 1. The dimension of the parts constituting the solid-state imaging device 10 may be optional. The same applies to dimensions in sectional views other than FIG. 1. The solid-state imaging device 10 irradiates an object 900 with light generated by a light source 800. The solid-state imaging device 10 receives light reflected by the object 900.

As shown in FIG. 1, the solid-state imaging device 10 includes a first layer 100, a second layer 200, a plurality of first microlenses 300, and a plurality of second microlenses 310. The first layer 100 includes a first principal surface 100a, a second principal surface 100b, a plurality of photoelectric conversion elements 110, and a first light transmission layer 120. The first principal surface 100a and the second principal surface 100b face opposite directions. The plurality of photoelectric conversion elements 110 receive light L2 incident on the first principal surface 100a. The first light transmission layer 120 allows light L1, which is incident on a second region S2 in the second principal surface 100b different from a first region S1 in the second principal surface 100b corresponding to the plurality of photoelectric conversion elements 110, to be emitted from the first principal surface 100a. The second layer 200 includes a third principal surface 200a, a fourth principal surface 200b, a first light blocking film 210, and a second light transmission layer 220. The third principal surface 200a and the fourth principal surface 200b face opposite directions. The third principal surface 200a faces the second principal surface 100b. The first light blocking film 210 is arranged in a third region S3 of the second layer 200 corresponding to the plurality of photoelectric conversion elements 110 and blocks the light L1 incident on the fourth principal surface 200b. The second light transmission layer 220 allows light, which is incident on a fifth region S5 in the fourth principal surface 200b that is different from a fourth region S4 in the fourth principal surface 200b corresponding to the first light blocking film 210 and that corresponds to the first light transmission layer 120, to be emitted from the third principal surface 200a. The plurality of first microlenses 300 are arranged in a sixth region S6 in the first principal surface 100a corresponding to the plurality of photoelectric conversion elements 110 and have a convex shape toward an outer side of the first principal surface 100a. The plurality of second microlenses 310 are arranged in the fifth region S5 on the fourth principal surface 200b and have a convex shape toward an outer side of the fourth principal surface 200b.

Details of the configuration shown in FIG. 1 will be described. The first layer 100 and the second layer 200 are stacked in a thickness direction Dr1 of the first layer 100. The thickness direction Dr1 of the first layer 100 is a direction perpendicular to the first principal surface 100a.

The first principal surface 100a and the second principal surface 100b are relatively wide surfaces among a plurality of surfaces constituting the surface of the first layer 100. The sixth region S6 of the first principal surface 100a overlaps the plurality of photoelectric conversion elements 110. The first region S1 of the second principal surface 100b overlaps the plurality of photoelectric conversion elements 110. The second region S2 of the second principal surface 100b overlaps the first light transmission layer 120.

FIG. 1 shows signs of one photoelectric conversion element 110 and one first light transmission layer 120 as representatives. The plurality of photoelectric conversion elements 110 (photodiodes) are configured with a semiconductor material. For example, the semiconductor material constituting the plurality of photoelectric conversion elements 110 includes at least one of silicon (Si), germanium (Ge), gallium (Ga), arsenic (As), and boron (B). The plurality of photoelectric conversion elements 110 convert light into signals. Some of the plurality of photoelectric conversion elements 110 may function in order to measure intensity of light. That is, the light L1 incident on the first light transmission layer 120 may be directly incident on only some of the plurality of photoelectric conversion elements 110.

For example, the first light transmission layer 120 is configured with a semiconductor material having an impurity concentration lower than that of the semiconductor material constituting the plurality of photoelectric conversion elements 110. In order to prevent electric charge generated by the light L1 incident on the first light transmission layer 120 from moving to the photoelectric conversion elements 110, element isolation may be formed between the photoelectric conversion elements 110 and the first light transmission layer 120. For example, a shallow trench isolation (STI) or a deep trench isolation (DTI) may be used as the element isolation. Alternatively, element isolation due to impurity implantation may also be used. The first light transmission layer 120 may also be configured with materials other than the semiconductor material. The plurality of photoelectric conversion elements 110 and the first light transmission layer 120 are alternately arranged in a direction Dr2 parallel to the first principal surface 100a.

The plurality of photoelectric conversion elements 110 constitute a part of the first principal surface 100a and a part of the second principal surface 100b. The first light transmission layer 120 may cover an upper side of the plurality of photoelectric conversion elements 110, so that the first principal surface 100a may be configured with only the first light transmission layer 120. The first light transmission layer 120 may cover a lower side of the plurality of photoelectric conversion elements 110, so that the second principal surface 100b may be configured with only the first light transmission layer 120.

The third principal surface 200a and the fourth principal surface 200b are relatively wide surfaces among a plurality of surfaces constituting the surface of the second layer 200. The third principal surface 200a contacts with the second principal surface 100b. The fourth region S4 of the fourth principal surface 200b overlaps the first light blocking film 210. The fifth region S5 of the fourth principal surface 200b overlaps the second light transmission layer 220.

FIG. 1 shows a sign of one first light blocking film 210 as a representative. The first light blocking film 210 is a thin film and is arranged in the vicinity of the fourth principal surface 200b. The position of the first light blocking film 210 is not limited to the position shown in FIG. 1. For example, the first light blocking film 210 may be arranged in the vicinity of the third principal surface 200a. The first light blocking film 210 may be in contact with the plurality of photoelectric conversion elements 110. The first light blocking film 210 is configured with a material having a light blocking property. The first light blocking film 210 may be configured with a metal such as copper (Cu), aluminum (Al), and tungsten (W).

The second light transmission layer 220 occupies parts of the second layer 200, other than the first light blocking film 210. The second light transmission layer 220 is configured with an insulating material. For example, the insulating material constituting the second light transmission layer 220 includes at least one of a silicon oxide film (SiO₂), a silicon nitride film (SiN), a silicon oxinitride film (SiON), a silicon oxycarbide film (SiOC), and a silicon carbonitride film (SiCN). The third region S3 of the second layer 200 overlaps the plurality of photoelectric conversion elements 110.

FIG. 1 shows signs of one first microlens 300 and one second microlens 310 as representatives. The plurality of first microlenses 300 are in contact with the first principal surface 100a. The plurality of first microlenses 300 are arranged on a side of the plurality of photoelectric conversion elements 110, which faces the object 900. The plurality of second microlenses 310 are in contact with the fourth principal surface 200b. The plurality of second microlenses 310 are arranged on a side of the first light blocking film 210, which faces the light source 800.

The plurality of first microlenses 300 are arranged only in the sixth region S6 on the first principal surface 100a. The plurality of second microlenses 310 are arranged only in the fifth region S5 on the fourth principal surface 200b.

For example, the focal position of each first microlens 300 exists inside each photoelectric conversion element 110. The focal position of each first microlens 300 may exist on a lower end side of each photoelectric conversion element 110, which faces the light source 800. For example, the focal position of each second microlens 310 exists on a side of the second principal surface 100b, which faces the object 900. The focal position of each second microlens 310 may exist on a lower end side of each photoelectric conversion element 110, which faces the object 900.

Some of the light L1 from the light source 800 is incident on the fourth principal surface 200b and is blocked by the first light blocking film 210. Therefore, the light L1 from the light source 800 is less likely to be directly incident on the photoelectric conversion elements 110. Some of the light L1 from the light source 800 passes through the second microlenses 310 and is incident on the fourth principal surface 200b. The light L1 incident on the fourth principal surface 200b is less likely to hit the first light blocking film 210 and is less likely to be directly incident on the photoelectric conversion elements 110 due to light gathering capability of the second microlenses 310. Therefore, light which is not irradiated to the object 900 and is incident on the photoelectric conversion elements 110 is reduced. The light L1 having passed through the second light transmission layer 220 by the second microlenses 310 is incident on the second principal surface 100b. The light L1 incident on the second principal surface 100b passes through the first light transmission layer 120 and is irradiated to the object 900 from the first principal surface 100a. The light L2 reflected by the object 900 passes through the first microlenses 300 and is incident on the first principal surface 100a. The light L2 incident on the first principal surface 100a is incident on the plurality of photoelectric conversion elements 110.

For example, a width D1 of the first light transmission layer 120 arranged between two adjacent photoelectric conversion elements 110 is the same as an interval D2 between the first light blocking films 210 respectively corresponding to the two adjacent photoelectric conversion elements 110. The width D1 may be larger than the interval D2. In this way, the light L1 passed through the second microlenses 310 and incident on the fourth principal surface 200b is less likely to be incident on the photoelectric conversion elements 110. The width D1 and the interval D2 are a dimension of a direction Dr2 parallel to the first principal surface 100a.

For example, a width D3 of the first light blocking film 210 is the same as a width D4 of each of the photoelectric conversion elements 110. The width D3 may be larger than the width D4. In this way, the light L1 from the light source 800 is less likely to be incident on the photoelectric conversion elements 110. The width D3 and the width D4 are the dimension of the direction Dr2 parallel to the first principal surface 100a.

For example, a diameter D5 of each of the first microlenses 300 is the same as the width D4 of each of the photoelectric conversion elements 110. The diameter D5 may be larger than the width D4. In this way, the solid-state imaging device 10 can efficiently receive the light L2 from the object 900 in the photoelectric conversion elements 110. The diameter D5 is the dimension of the direction Dr2 parallel to the first principal surface 100a.

For example, a diameter D6 of each of the second microlenses 310 is the same as the width D1 of the first light transmission layer 120 and the interval D2 between the first light blocking films 210. The diameter D6 may be larger than the width D1 and the interval D2. In this way, the solid-state imaging device 10 can efficiently irradiate the object 900 with the light L1 from the light source 800. The diameter D6 is the dimension of the direction Dr2 parallel to the first principal surface 100a.

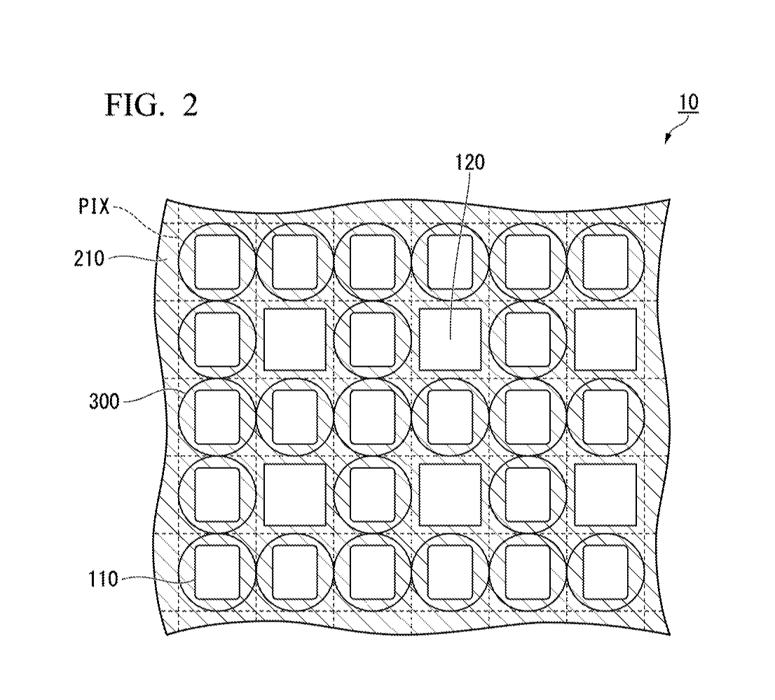

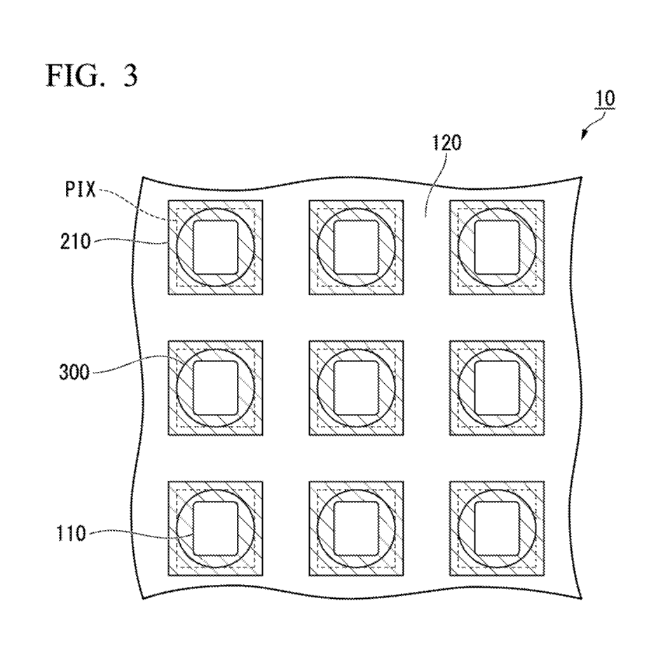

FIG. 2 and FIG. 3 show the positions of the plurality of photoelectric conversion elements 110, the plurality of first microlenses 300, the first light transmission layer 120, and the first light blocking film 210. FIG. 2 shows a first example and FIG. 3 shows a second example. FIG. 2 and FIG. 3 show states in which the solid-state imaging device 10 is viewed in a direction perpendicular to the first principal surface 100a. That is. FIG. 2 and FIG. 3 show states in which the solid-state imaging device 10 is viewed from the front surface of the first layer 100. The first light blocking film 210 is arranged inside the second layer 200. However, in FIG. 2 and FIG. 3, the first light blocking film 210 is transparently shown.

FIG. 2 and FIG. 3 show signs of one photoelectric conversion element 110, one first microlens 300, and one first light transmission layer 120 as representatives. The plurality of photoelectric conversion elements 110, the plurality of first microlenses 300, and the first light transmission layer 120 are arranged in a matrix form. Each of the plurality of photoelectric conversion elements 110 constitutes one pixel PIX. The solid-state imaging device 10 has a plurality of pixels PIX. FIG. 2 and FIG. 3 show a sign of one pixel PIX as a representative. The plurality of pixels PIX are arranged in a matrix form. The plurality of second microlenses 310 not shown in FIG. 2 and FIG. 3 overlap the plurality of first microlenses 300.

When the solid-state imaging device 10 is viewed in the direction perpendicular to the first principal surface 100a, each of the plurality of photoelectric conversion elements 110 overlaps any one of the plurality of first microlenses 300 and any one of the plurality of second microlenses 310. One photoelectric conversion element 110 and one first microlens 300 correspond to each other. One photoelectric conversion element 110 and one second microlens 310 correspond to each other. When the solid-state imaging device 10 is viewed in the direction perpendicular to the first principal surface 100a, the center of the photoelectric conversion element 110 and the center of the first microlens 300 coincide with each other. When the solid-state imaging device 10 is viewed in the direction perpendicular to the first principal surface 100a, the center of the photoelectric conversion element 110 and the center of the second microlens 310 coincide with each other. In FIG. 2, the first light blocking film 210 is configured with one thin film and has a plurality of openings. In FIG. 2, the first light transmission layer 120 is arranged in a region corresponding to the openings of the first light blocking film 210. In FIG. 3, a plurality of first light blocking films 210 are arranged.

The solid-state imaging device 10 according to the first embodiment includes the plurality of second microlenses 310 arranged in the fifth region S5 in the fourth principal surface 200b. The fifth region S5 is different from the fourth region S4 in the fourth principal surface 200b corresponding to the first light blocking film 210 and corresponds to the first light transmission layer 120. Therefore, the solid-state imaging device 10 can reduce light which is not irradiated to the object 900 and is incident on the photoelectric conversion elements 110.

### Second Embodiment

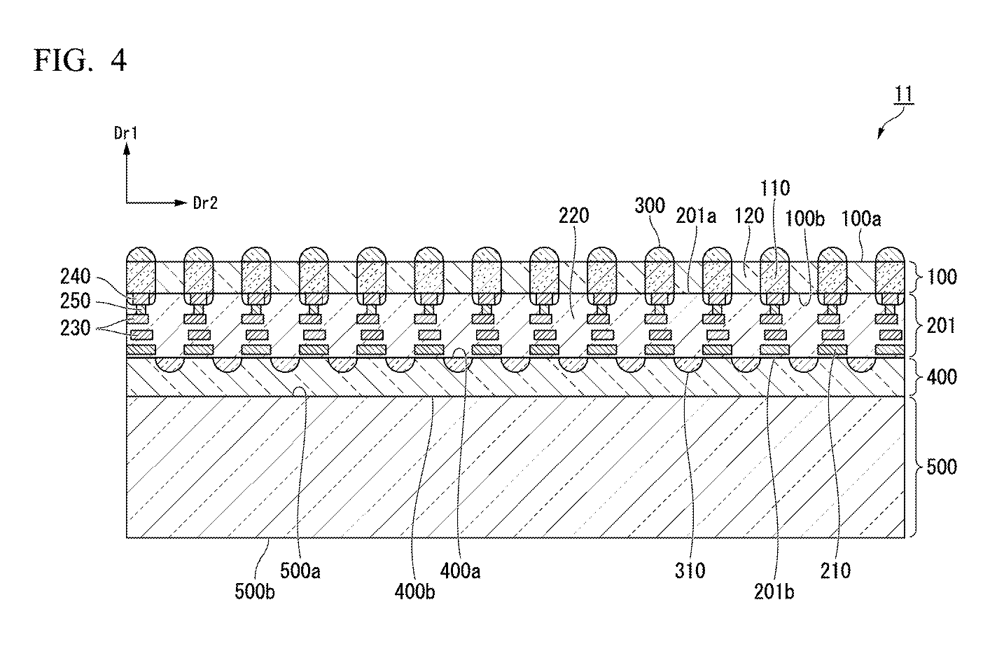

FIG. 4 shows a configuration of a solid-state imaging device 11 according to a second embodiment of the present invention. FIG. 4 shows a section of the solid-state imaging device 11. The configuration shown in FIG. 4, which is different from that shown in FIG. 1, will be described.

The second layer 200 of the solid-state imaging device 10 shown in FIG. 1 is changed to a second layer 201. The second layer 201 includes a third principal surface 201a, a fourth principal surface 201b, the first light blocking film 210, the second light transmission layer 220, wirings 230, a plurality of transistors 240, and a plurality of vias 250. FIG. 4 shows signs of one first light blocking film 210, one wiring 230, one transistor 240, and one via 250 as representatives.

The third principal surface 201a is configured similarly to the third principal surface 200a in the solid-state imaging device 10 shown in FIG. 1. The fourth principal surface 201b is configured similarly to the fourth principal surface 200b in the solid-state imaging device 10 shown in FIG. 1.

The second layer 201 (a wiring layer) includes a plurality of wirings 230 having a metal. For example, a main material of the wiring 230 is a metal such as copper (Cu), aluminum (Al), and tungsten (W). The wiring 230 may include at least one of titanium (Ti), tantalum (Ta), and chrome (Cr) or nitrides thereof. The wiring 230 is a thin film in which a wiring pattern has been formed. The wiring 230 transmits signals generated by the photoelectric conversion elements 110. Only one layer of the wiring 230 may be arranged, or a plurality of layers of the wiring 230 may be arranged. In the example shown in FIG. 4, two layers of the wiring 230 are arranged.

The first light blocking film 210 may include a conductive material. A power supply voltage or a ground voltage may be applied to the first light blocking film 210. The conductive material constituting the first light blocking film 210 may be the same as the material constituting the wiring 230. When a predetermined voltage is applied to the first light blocking film 210, an influence to the wiring 230 from the first light blocking film 210 is reduced. The first light blocking film 210 can serve as a power supply wiring or a ground wiring. The first light blocking film 210 may be a part of the wiring 230.

The plurality of transistors 240 are electrically connected to the plurality of photoelectric conversion elements 110 and are arranged between the plurality of photoelectric conversion elements 110 and the fourth principal surface 201b. FIG. 4 shows only gate electrodes of the transistors 240. Each of the transistors 240 has a source electrode and a drain electrode. However, in FIG. 4, the source electrode and the drain electrode are not shown.

Each of the plurality of transistors 240 is connected to each of the vias 250. The vias 250 are connected to the wirings 230. Consequently, the plurality of transistors 240 are electrically connected to the wirings 230. The plurality of transistors 240 read signals generated by the plurality of photoelectric conversion elements 110 and output the read signals to the wirings 230. For example, a material constituting the vias 250 is the same as the material constituting the wirings 230. The wirings 230 of a different layer are connected by the vias which are the same as the vias 250.

The solid-state imaging device 11 further includes a third layer 400 and a support substrate 500. The third layer 400 includes the plurality of second microlenses 310. The third layer 400 faces the fourth principal surface 201b. The third layer 400 is arranged between the second layer 201 and the support substrate 500. The third layer 400 allows light incident on the third layer 400 to pass therethrough. The support substrate 500 includes a fifth principal surface 500a and a sixth principal surface 500b and allows light incident on the support substrate 500 to pass therethrough. The fifth principal surface 500a and the sixth principal surface 500b face opposite directions. The fifth principal surface 500a faces the third layer 400.

The first layer 100, the second layer 201, the third layer 400, and the support substrate 500 are stacked in the thickness direction Dr1 of the first layer 100. The third layer 400 includes a seventh principal surface 400a and an eighth principal surface 400b. The seventh principal surface 400a and the eighth principal surface 400b are relatively wide surfaces among a plurality of surfaces constituting the surface of the third layer 400. The seventh principal surface 400a and the eighth principal surface 400b face opposite directions. The seventh principal surface 400a faces the fourth principal surface 201b and contacts with the fourth principal surface 201b. For example, the third layer 400 is configured with a resin adhesive or an inorganic thin film. For example, the resin adhesive constituting the third layer 400 is a highly heat-resistant organic adhesive using benzocyclobutene as a main material. For example, the inorganic thin film constituting the third layer 400 includes at least one of a silicon oxide film (SiO₂), a silicon nitride film (SiN), a silicon oxinitride film (SiON), a silicon oxycarbide film (SiOC), and a silicon carbonitride film (SiCN). The third layer 400 allows light incident on the eighth principal surface 400b to be emitted from the seventh principal surface 400a. For example, the third layer 400 and the support substrate 500 are bonded to each other by plasma surface activation bonding or directly bonding.

The fifth principal surface 500a and the sixth principal surface 500b are relatively wide surfaces of a plurality of surfaces constituting the surface of the support substrate 500. The fifth principal surface 500a faces the eighth principal surface 400b and contacts with the eighth principal surface 400b. The support substrate 500 is configured with a transparent material. For example, the transparent material constituting the support substrate 500 is glass. The support substrate 500 allows light incident on the sixth principal surface 500b to be emitted from the fifth principal surface 500a. The support substrate 500 may be configured such that light incident on a side surface of the support substrate 500 is emitted from the fifth principal surface 500a.

Light from the light source 800 is incident on the sixth principal surface 500b. The light incident on the sixth principal surface 500b passes through the support substrate 500 and is incident on the eighth principal surface 400b. The light incident on the eighth principal surface 400b is incident on the plurality of second microlenses 310.

A part of one photoelectric conversion element 110, one transistor 240, and the wiring 230 constitutes a pixel. The solid-state imaging device 11 may also include a driving circuit, a reading circuit, a signal processing circuit, an output circuit, and an electrode. The driving circuit drives the pixel. The reading circuit reads a signal from the pixel. The signal processing circuit processes the signal read from the pixel. The output circuit outputs a signal processed by the signal processing circuit to an exterior of the solid-state imaging device 11. The electrode is arranged on at least one of the first principal surface 100a and the sixth principal surface 500b. The electrode performs input/output of signals with the exterior of the solid-state imaging device 11. A wire may be connected to the electrode by a wire bonding method. A bump may be provided on the electrode by a bumping method.

The configuration shown in FIG. 4, other than the above, is similar to the configuration shown in FIG. 1.

The solid-state imaging device 11 need not include at least one of the wirings 230, the plurality of transistors 240, and the vias 250. The solid-state imaging device 11 need not include the third layer 400 and the support substrate 500, other than the plurality of second microlenses 310.

The solid-state imaging device 11 according to the second embodiment can reduce light which is not irradiated to the object 900 and is incident on the photoelectric conversion elements 110.

The plurality of transistors 240 are arranged between the plurality of photoelectric conversion elements 110 and the fourth principal surface 201b. As compared with a case where the plurality of transistors 240 are arranged on a side of the plurality of photoelectric conversion elements 110 facing the object 900, light incident on the plurality of photoelectric conversion elements 110 increases.

### Third Embodiment

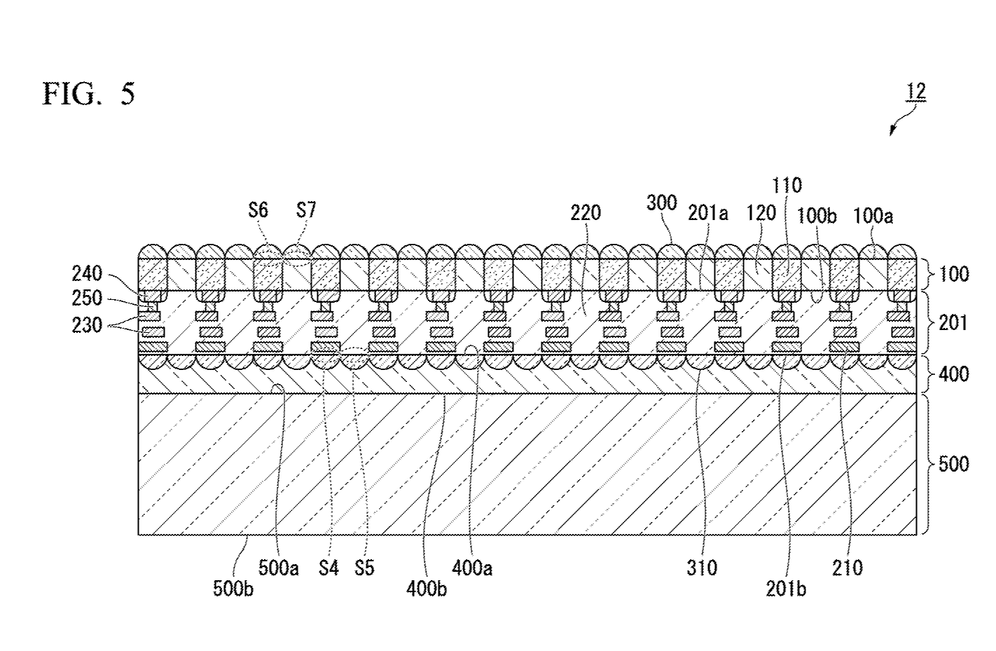

FIG. 5 shows a configuration of a solid-state imaging device 12 according to a third embodiment of the present invention. FIG. 5 shows a section of the solid-state imaging device 12. The configuration shown in FIG. 5, which is different from that shown in FIG. 4, will be described.

The plurality of first microlenses 300 are arranged in the sixth region S6, which corresponds to the plurality of photoelectric conversion elements 110, and a seventh region S7 of the first principal surface 100a which corresponds to the first light transmission layer 120. The sixth region S6 of the first principal surface 100a overlaps the plurality of photoelectric conversion elements 110. The seventh region S7 of the first principal surface 100a overlaps the first light transmission layer 120.

The plurality of second microlenses 310 are arranged in the fifth region S5, which corresponds to the first light transmission layer 120, and the fourth region S4, which corresponds to the first light blocking film 210, on the fourth principal surface 201b. The fifth region S5 of the fourth principal surface 201b overlaps the first light transmission layer 120. The fourth region S4 of the fourth principal surface 201b overlaps the first light blocking film 210.

The plurality of first microlenses 300 may be arranged in the sixth region S6 and the seventh region S7 and the plurality of second microlenses 310 may be arranged only in the fifth region S5. The plurality of second microlenses 310 may be arranged in the fifth region S5 and the fourth region S4 and the plurality of first microlenses 300 may be arranged only in the sixth region S6.

The configuration shown in FIG. 5, other than the above, is similar to the configuration shown in FIG. 4.

The solid-state imaging device 12 need not include at least one of the wirings 230, the plurality of transistors 240, and the vias 250. The solid-state imaging device 12 need not include the third layer 400 and the support substrate 500, other than the plurality of second microlenses 310.

The solid-state imaging device 12 according to the third embodiment can reduce light which is not irradiated to the object 900 and is incident on the photoelectric conversion elements 110.

The plurality of first microlenses 300 are arranged in the seventh region S7, so that the solid-state imaging device 12 can efficiently irradiate the object 900 with light having passed through the first light transmission layer 120. The plurality of second microlenses 310 are arranged in the fourth region S4, so that the solid-state imaging device 12 can efficiently block unnecessary light by the first light blocking film 210.

### Fourth Embodiment

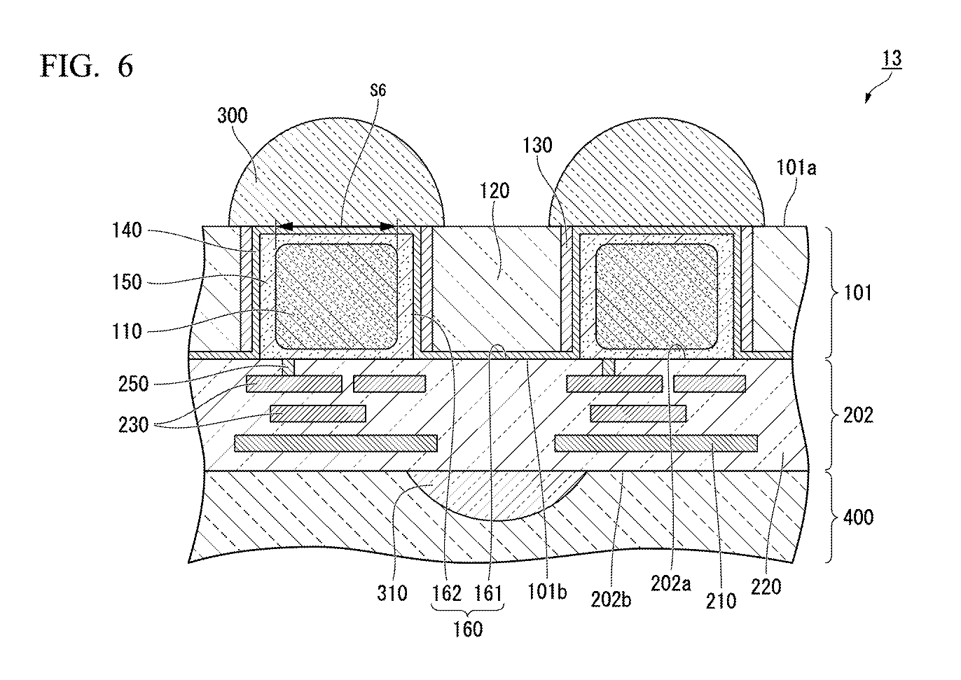

FIG. 6 shows a configuration of a solid-state imaging device 13 according to a fourth embodiment of the present invention. FIG. 6 shows a partial section of the solid-state imaging device 13. In FIG. 6, a part of the third layer 400 and the support substrate 500 are not shown. The configuration shown in FIG. 6, which is different from that shown in FIG. 4, will be described.

The first layer 100 in the solid-state imaging device 11 shown in FIG. 4 is changed to a first layer 101. The first layer 101 includes a first principal surface 101a, a second principal surface 101b, the plurality of photoelectric conversion elements 110, the first light transmission layer 120, a second light blocking film 130, an antireflection film 140, a semiconductor layer 150, and a groove 160. FIG. 6 shows signs of one photoelectric conversion element 110, one first light transmission layer 120, one second light blocking film 130, and one semiconductor layer 150 as representatives.

The first principal surface 101a is configured similarly to the first principal surface 100a in the solid-state imaging device 11 shown in FIG. 4. The second principal surface 101b is configured similarly to the second principal surface 100b in the solid-state imaging device 11 shown in FIG. 4.

The first light transmission layer 120 is arranged in the groove 160 formed between the plurality of photoelectric conversion elements 110. The groove 160 is a region formed by removing a part of the first layer 101. The groove 160 has a bottom surface 161 and a side surface 162. In FIG. 6, the groove 160 passes through the first layer 101. Therefore, the bottom surface 161 serves as a third principal surface 202a of a second layer 202. The groove 160 need not pass through the first layer 101.

The first light transmission layer 120 is configured with a transparent material filled in the groove 160. The transparent material constituting the first light transmission layer 120 is a material having a light absorption rate smaller than that of a semiconductor material. For example, the transparent material constituting the first light transmission layer 120 is a transparent resin such as a novolac resin. The transparent material constituting the first light transmission layer 120 may include at least one of an inorganic material, a silicon oxide film (SiO₂), and a silicon nitride film (SiN). When the silicon oxide film is used as the first light transmission layer 120, the silicon oxide film is formed and then a surface of the silicon oxide film is planarized by a surface planarization technology such as a chemical mechanical polishing (CMP).

The second light blocking film 130 is arranged on the side surface 162 of the groove 160 and blocks light incident on the first light transmission layer 120. The second light blocking film 130 covers the side surface 162. Specifically, the second light blocking film 130 covers the antireflection film 140 arranged on the side surface 162. The second light blocking film 130 is configured with a material having a light blocking property. For example, a main material of the second light blocking film 130 is a metal such as copper (Cu), aluminum (Al), and tungsten (W). The second light blocking film 130 may include at least one of titanium (Ti), tantalum (Ta), and chrome (Cr) or nitrides thereof.

The antireflection film 140 is arranged on the bottom surface 161 and the side surface 162 of the groove 160. The antireflection film 140 is arranged in the sixth region S6 corresponding to the photoelectric conversion element 110 on the first principal surface 101a. The antireflection film 140 constitutes a part of the first principal surface 101a. The antireflection film 140 is configured with a thin film-like high dielectric material having a thickness of several tens of nm or more and 100 nm or less. For example, the high dielectric material constituting the antireflection film 140 includes at least one of titanium oxide (TiO₂), tantalum oxide (TaO), hafnium oxide (HfO), and a silicon nitride film (SiN). The high dielectric material constituting the antireflection film 140 may include an organic material having a high refractive index. The antireflection film 140 prevents reflection of light incident on the first principal surface 101a.

The semiconductor layer 150 is arranged in a region corresponding to the photoelectric conversion elements 110 in the first layer 101. The photoelectric conversion elements 110 are arranged inside the semiconductor layer 150. For example, the semiconductor layer 150 is configured with a semiconductor material having an impurity concentration lower than that of the semiconductor material constituting the plurality of photoelectric conversion element 110.

The second layer 201 in the solid-state imaging device 11 shown in FIG. 4 is changed to a second layer 202. The second layer 202 includes a third principal surface 202a, a fourth principal surface 202b, the first light blocking film 210, the second light transmission layer 220, the wirings 230, and the plurality of vias 250. FIG. 6 shows signs of one first light blocking film 210, one wiring 230, and one via 250 as representatives.

The third principal surface 202a is configured similarly to the third principal surface 201a in the solid-state imaging device 11 shown in FIG. 4. The fourth principal surface 202b is configured similarly to the fourth principal surface 201b in the solid-state imaging device 11 shown in FIG. 4.

The second layer 202 does not include the transistors 240 in the solid-state imaging device 11 shown in FIG. 4. The plurality of photoelectric conversion elements 110 are electrically connected to the wirings 230 by the vias 250.

The focal distance of each of a plurality of first microlenses 300 is shorter than that of each of a plurality of second microlenses 310. For example, a curvature radius of the first microlens 300 is set to be smaller than that of the second microlens 310. A difference between the refractive indexes of the first microlens 300 and the semiconductor layer 150 may be set to be larger than that between the refractive indexes of the second microlens 310 and the second light transmission layer 220.

The configuration shown in FIG. 6, other than the above, is similar to the configuration shown in FIG. 4.

The solid-state imaging device 13 need not include the plurality of transistors 240. The solid-state imaging device 13 need not include at least one of the antireflection film 140, the semiconductor layer 150, the wirings 230, and the plurality of vias 250. The solid-state imaging device 13 need not include the third layer 400 and the support substrate 500 other than the plurality of second microlenses 310. The plurality of first microlenses 300 may be similar to the plurality of first microlenses 300 in the solid-state imaging device 12 shown in FIG. 5. The plurality of second microlenses 310 may be similar to the plurality of second microlenses 310 in the solid-state imaging device 12 shown in FIG. 5.

The solid-state imaging device 13 according to the fourth embodiment can reduce light which is not irradiated to the object 900 and is incident on the photoelectric conversion elements 110.

Since the first light transmission layer 120 is arranged in the groove 160, the first light transmission layer 120 can be configured with a material other than a semiconductor material. The first light transmission layer 120 is configured with a transparent material having a light absorption rate smaller than that of the semiconductor material, resulting in an increase in light irradiated to the object 900. As a consequence, light incident on the plurality of photoelectric conversion elements 110 increases.

Since the second light blocking film 130 is arranged on the side surface 162 of the groove 160, light incident on the first light transmission layer 120 is less likely to be incident on the photoelectric conversion elements 110.

The focal distance of each of the plurality of first microlenses 300 is shorter than that of each of the plurality of second microlenses 310. Therefore, the solid-state imaging device 13 can efficiently irradiate the object 900 with light having passed through the first light transmission layer 120, and can efficiently receive light from the object 900 through the photoelectric conversion elements 110.

### Fifth Embodiment

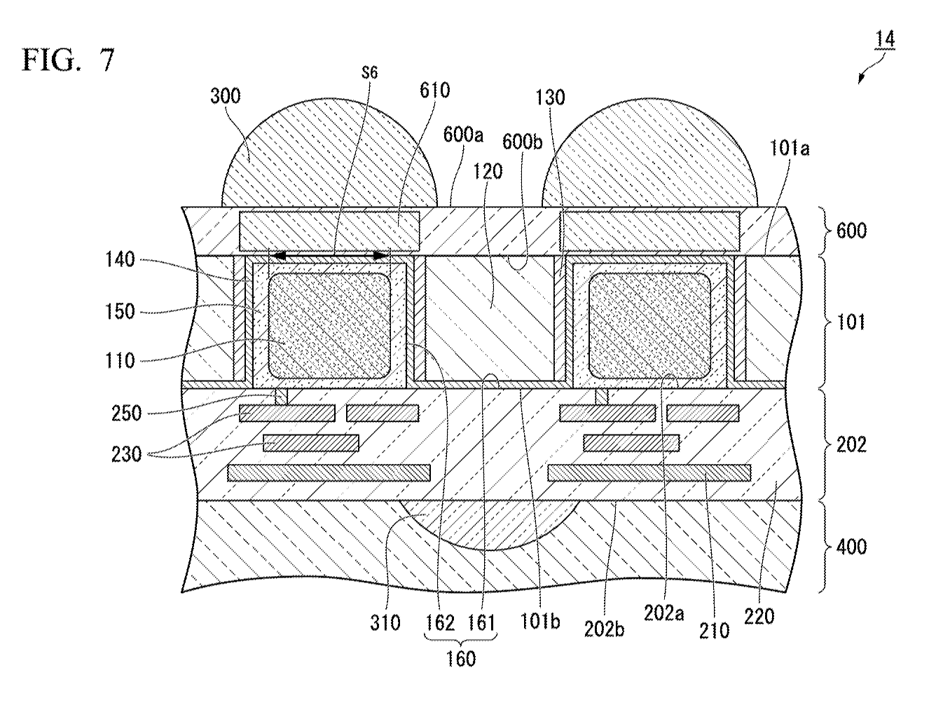

FIG. 7 shows a configuration of a solid-state imaging device 14 according to a fifth embodiment of the present invention. FIG. 7 shows a partial section of the solid-state imaging device 14. In FIG. 7, a part of the third layer 400 and the support substrate 500 are not shown. The configuration shown in FIG. 7, which is different from that shown in FIG. 6, will be described.

The solid-state imaging device 14 further includes a filter layer 600. The filter layer 600 is arranged between the plurality of photoelectric conversion elements 110 and the plurality of first microlenses 300. The filter layer 600 includes a ninth principal surface 600a, a tenth principal surface 600b, and a plurality of color filters 610. The ninth principal surface 600a and the tenth principal surface 600b are relatively wide surfaces among a plurality of surfaces constituting the surface of the filter layer 600. The ninth principal surface 600a and the tenth principal surface 600b face opposite directions. The plurality of first microlenses 300 are arranged on the ninth principal surface 600a and in contact with the ninth principal surface 600a. The tenth principal surface 600b faces the first principal surface 101a and is in contact with the first principal surface 101a.

The plurality of color filters 610 are arranged in the sixth region S6, which corresponds to the plurality of photoelectric conversion element 110, on the first principal surface 101a, and are arranged between the plurality of photoelectric conversion elements 110 and the plurality of first microlenses 300. For example, the filter layer 600 is configured with a transparent material. For example, the transparent material constituting the plurality of color filters 610 is a transparent resin to which pigment for absorbing light of a predetermined wavelength range is added.

Light reflected by the object 900 passes through the first microlenses 300 and is incident on the ninth principal surface 600a. The light incident on the ninth principal surface 600a is incident on the plurality of color filters 610. The plurality of color filters 610 allow only visible light which has a wavelength corresponding to a predetermined color to pass therethrough. The light having passed through the plurality of color filters 610 is incident on the first principal surface 101a. The light incident on the first principal surface 101a passes through the antireflection film 140 and the semiconductor layer 150 and is incident on the plurality of photoelectric conversion element 110.

The configuration shown in FIG. 7, other than the above, is similar to the configuration shown in FIG. 6.

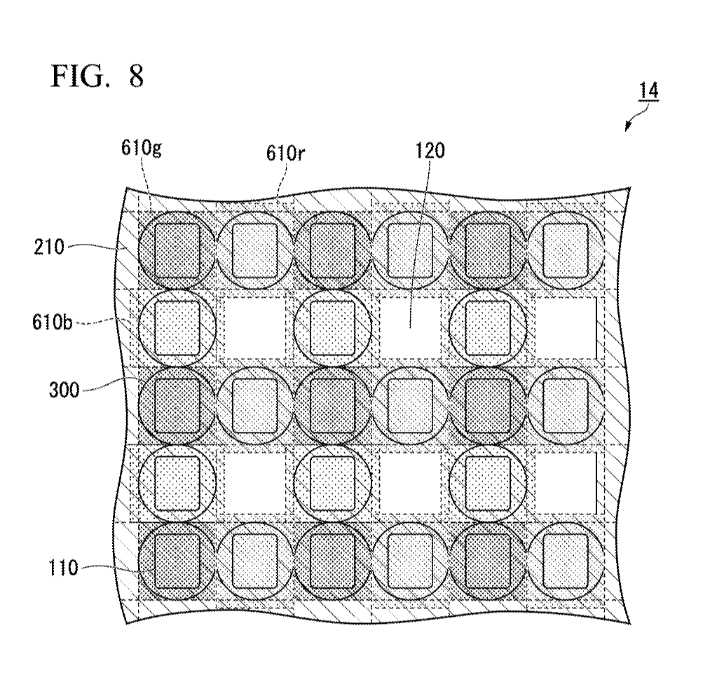

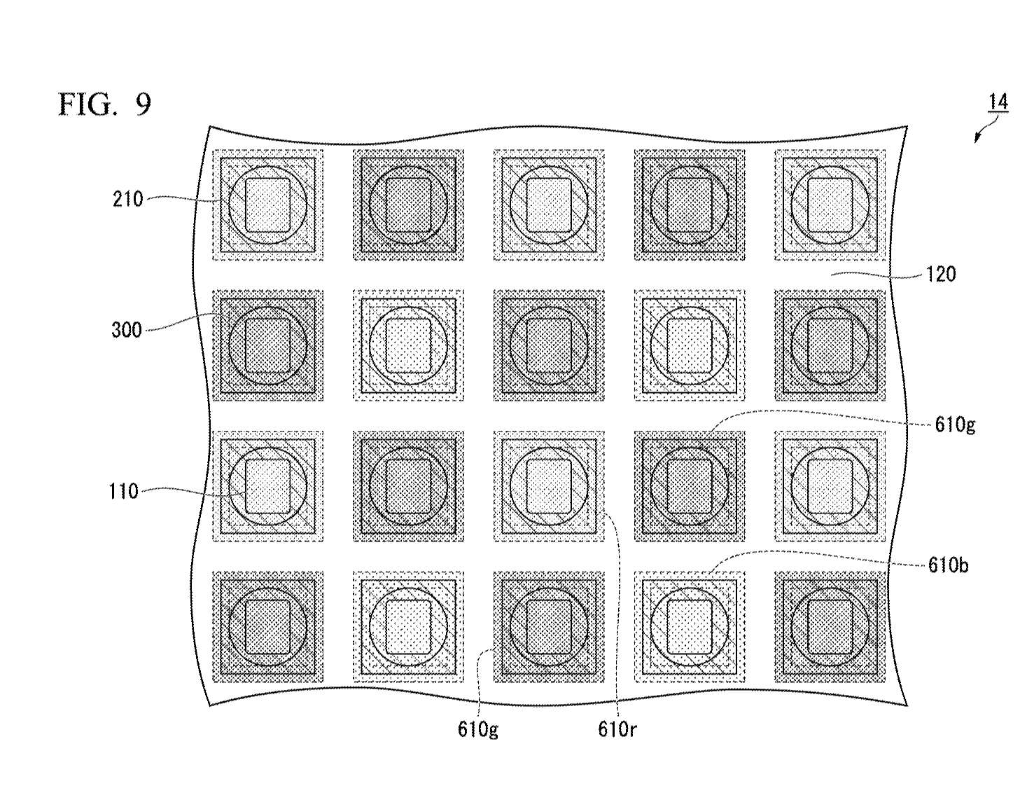

FIG. 8 and FIG. 9 show the positions of the plurality of photoelectric conversion element 110, the plurality of first microlenses 300, the first light transmission layer 120, the first light blocking film 210, and the plurality of color filters 610. FIG. 8 shows a first example and FIG. 9 shows a second example. FIG. 8 and FIG. 9 show states when the solid-state imaging device 14 is viewed in a direction perpendicular to the first principal surface 101a. That is. FIG. 8 and FIG. 9 show states when the solid-state imaging device 14 is viewed from the front surface of the first layer 101. The first light blocking film 210 is arranged inside the second layer 202. However, in FIG. 8 and FIG. 9, the first light blocking film 210 is transparently shown.

In FIG. 8 and FIG. 9, the plurality of color filters 610 include color filters 610r, color filters 610g, and color filters 610b. FIG. 8 and FIG. 9 show signs of one color filter 610r, one color filter 610g, and one color filter 610b as representatives. The color filter 610r allows only light which has a wavelength corresponding to red to pass therethrough. The color filter 610g allows only light which has a wavelength corresponding to green to pass therethrough. The color filter 610b allows only light which has a wavelength corresponding to blue to pass therethrough.

In FIG. 8, the positions of the plurality of photoelectric conversion elements 110, the plurality of first microlenses 300, the first light transmission layer 120, and the first light blocking film 210 are similar to those of the elements shown in FIG. 2. In FIG. 9, the positions of the plurality of photoelectric conversion elements 110, the plurality of first microlenses 300, the first light transmission layer 120, and the first light blocking film 210 are similar to those of the elements shown in FIG. 3. In FIG. 8 and FIG. 9, the color filters 610r, the color filters 610g, and the color filters 610b are arranged in a matrix form.

When the solid-state imaging device 14 is viewed in the direction perpendicular to the first principal surface 101a, each of the plurality of photoelectric conversion elements 110 overlaps any one of the plurality of color filters 610. One photoelectric conversion element 110 corresponds to one color filter 610. When the solid-state imaging device 14 is viewed in the direction perpendicular to the first principal surface 101a, the center of the photoelectric conversion elements 110 and the center of the color filters 610 coincide with each other. The photoelectric conversion element 110, on which light having passed through the color filter 610r is incident, generates a signal corresponding to red. The photoelectric conversion element 110, on which light having passed through the color filter 610g is incident, generates a signal corresponding to green. The photoelectric conversion element 110, on which light having passed through the color filter 610b is incident, generates a signal corresponding to blue.

The solid-state imaging device 14 may include the plurality of transistors 240. The solid-state imaging device 14 need not include at least one of the antireflection film 140, the semiconductor layer 150, the wirings 230, and the vias 250. The solid-state imaging device 14 need not include the third layer 400 and the support substrate 500 other than the plurality of second microlenses 310. The plurality of first microlenses 300 may be similar to the plurality of first microlenses 300 in the solid-state imaging device 12 shown in FIG. 5. The plurality of second microlenses 310 may be similar to the plurality of second microlenses 310 in the solid-state imaging device 12 shown in FIG. 5.

The solid-state imaging device 14 according to the fifth embodiment can reduce light which is not irradiated to the object 900 and is incident on the photoelectric conversion elements 110.

Since the plurality of color filters 610 are arranged, the solid-state imaging device 14 can acquire color signals.

### Sixth Embodiment

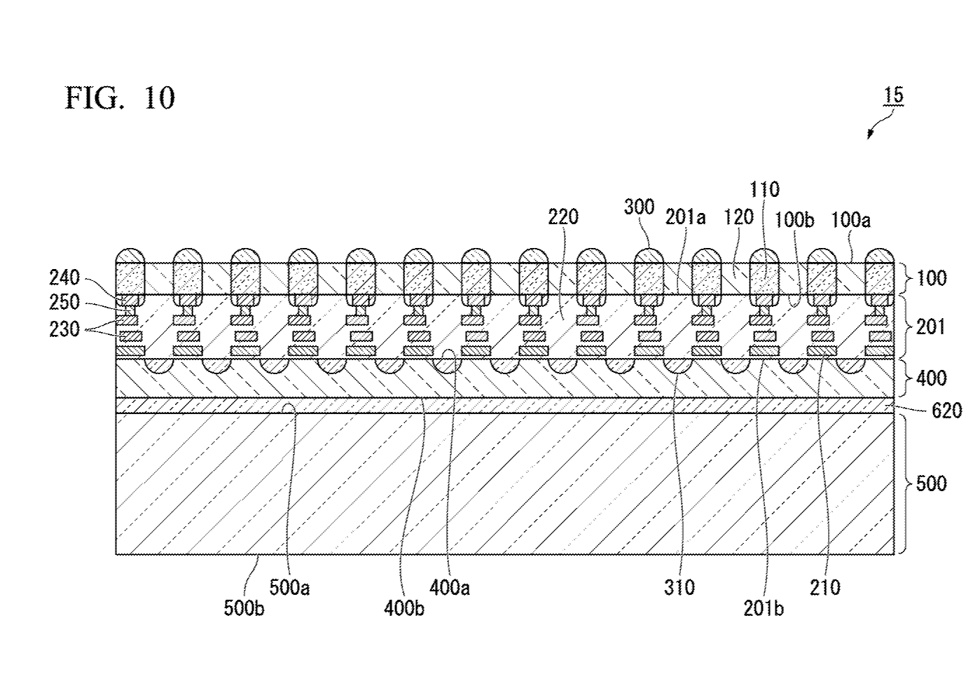

FIG. 10 shows a configuration of a solid-state imaging device 15 according to a sixth embodiment of the present invention. FIG. 10 shows a section of the solid-state imaging device 15. The configuration shown in FIG. 10, which is different from that shown in FIG. 4, will be described.

The solid-state imaging device 15 includes the third layer 400 and a filter 620. The third layer 400 includes the plurality of second microlenses 310. The third layer 400 faces the fourth principal surface 201b. The third layer 400 is arranged between the second layer 201 and the filter 620. The third layer 400 allows light incident on the third layer 400 to pass therethrough. The filter 620 has a structure in which a plurality of films including a dielectric are stacked.

The filter 620 is a thin film. The filter 620 faces the eighth principal surface 400b and is in contact with the eighth principal surface 400b. The filter 620 faces the fifth principal surface 500a and is in contact with the fifth principal surface 500a. For example, the filter 620 has a structure in which a film having a high dielectric material and a film having a low dielectric material are alternately stacked. For example, the high dielectric material constituting the filter 620 includes at least one of titanium oxide (TiO₂), tantalum oxide (TaO), hafnium oxide (HfO), and a silicon nitride film (SiN). The high dielectric material constituting the filter 620 may include an organic material having a high refractive index. The low dielectric material constituting the filter 620 includes a silicon oxide film (SiO₂). The low dielectric material constituting the filter 620 may include an organic material having a low refractive index.

Light incident on the sixth principal surface 500b passes through the support substrate 500 and is incident on the filter 620. The filter 620 allows only light corresponding to a predetermined wavelength to pass therethrough. The light having passed through the filter 620 is incident on the eighth principal surface 400b.

For example, the filter 620 allows special light to pass therethrough. For example, the special light includes fluorescence. In a medical site, a lesion part is observed using a color image and a fluorescent image. For example, exciting light is irradiated to an indocyanine green (ICG) and fluorescence from a lesion part is detected. The ICG is a fluorescent material. The ICG is administered in advance to a body of a person to be inspected. The ICG is excited in an infrared region by the exciting light and emits fluorescence. The administered ICG is accumulated at a lesion part of a cancer and the like. Since strong fluorescence is generated from the lesion part, an inspector can determine the presence or absence of the lesion part on the basis of a captured fluorescent image. The plurality of photoelectric conversion elements 110 generate signals based on the fluorescence.

The special light may include narrow-band light. Light having a wavelength which is easily absorbed in hemoglobin of blood is irradiated to a blood vessel, so that it is possible to acquire an image with an emphasized blood vessel. For example, blue narrow-band light or green narrow-band light is irradiated to a blood vessel. The plurality of photoelectric conversion elements 110 generate signals based on the narrow-band light.

The configuration shown in FIG. 10, other than the above, is similar to the configuration shown in FIG. 4.

The solid-state imaging device 15 need not include at least one of the wirings 230, the plurality of transistors 240, and the vias 250. The solid-state imaging device 15 need not include the support substrate 500. A plurality of first microlenses 300 may be similar to the plurality of first microlenses 300 in the solid-state imaging device 12 shown in FIG. 5. A plurality of second microlenses 310 may be similar to the plurality of second microlenses 310 in the solid-state imaging device 12 shown in FIG. 5. The first layer 100 may be changed to the first layer 101 in the solid-state imaging device 13 shown in FIG. 6.

The solid-state imaging device 15 according to the sixth embodiment can reduce light which is not irradiated to the object 900 and is incident on the photoelectric conversion elements 110.

Since the filter 620 is arranged, the solid-state imaging device 15 can acquire signals corresponding to light having a predetermined wavelength. The filter 620 is arranged on a side of the second microlenses 310, which faces the light source 800. Therefore, only light corresponding to a predetermined wavelength passes through the plurality of second microlenses 310 and is easily incident on the second light transmission layer 220. As a consequence, light, other than the light corresponding to the predetermined wavelength, is less likely to be directly incident on the photoelectric conversion elements 110. That is, in the photoelectric conversion elements 110, noise due to the light other than the light corresponding to the predetermined wavelength hardly occurs.

### Modification Example of Sixth Embodiment

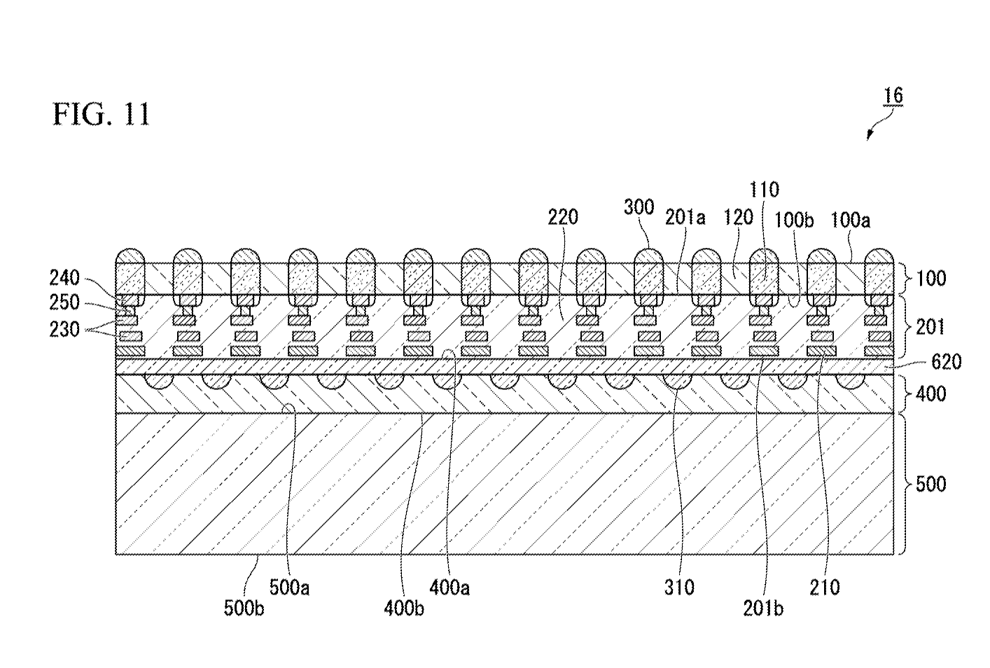

FIG. 11 shows a configuration of a solid-state imaging device 16 according to a modification example of the sixth embodiment of the present invention. FIG. 11 shows a section of the solid-state imaging device 16. The configuration shown in FIG. 11, which is different from that shown in FIG. 10, will be described.

The arrangement position of the filter 620 is different from that of the filter 620 in the solid-state imaging device 15 shown in FIG. 10. The filter 620 is arranged between the second layer 201 and the plurality of second microlenses 310. The filter 620 faces the fourth principal surface 201b and is in contact with the fourth principal surface 201b. The filter 620 faces the seventh principal surface 400a and is in contact with the seventh principal surface 400a.

Light having passed through the plurality of second microlenses 310 is incident on the filter 620. The filter 620 allows only light corresponding to a predetermined wavelength to pass therethrough. The light having passed through the filter 620 is incident on the fourth principal surface 201b.

The configuration shown in FIG. 11, other than the above, is similar to the configuration shown in FIG. 10.

The solid-state imaging device 16 need not include at least one of the wirings 230, the plurality of transistors 240, and the vias 250. The solid-state imaging device 16 need not include the third layer 400 and the support substrate 500 other than the plurality of second microlenses 310. A plurality of first microlenses 300 may be similar to the plurality of first microlenses 300 in the solid-state imaging device 12 shown in FIG. 5. A plurality of second microlenses 310 may be similar to the plurality of second microlenses 310 in the solid-state imaging device 12 shown in FIG. 5. The first layer 100 may be changed to the first layer 101 in the solid-state imaging device 13 shown in FIG. 6.

### Seventh Embodiment

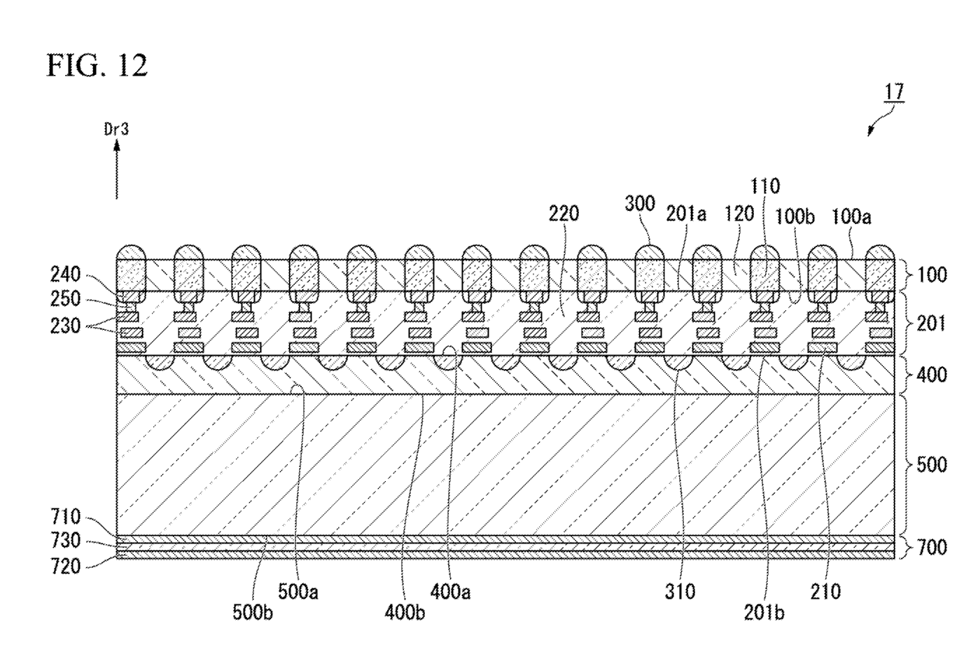

FIG. 12 shows a configuration of a solid-state imaging device 17 according to a seventh embodiment of the present invention. FIG. 12 shows a section of the solid-state imaging device 17. The configuration shown in FIG. 12, which is different from that shown in FIG. 4, will be described.

The solid-state imaging device 17 further includes a light emitting element 700. The light emitting element 700 includes a first electrode layer 710, a second electrode layer 720, and a light emitting layer 730. The first electrode layer 710, the second electrode layer 720, and the light emitting layer 730 are stacked in a thickness direction DR3 of the support substrate 500. The first electrode layer 710 faces the sixth principal surface 500b. The light emitting layer 730 is arranged between the first electrode layer 710 and the second electrode layer 720.

The light emitting element 700 includes a light source. The thickness direction DR3 of the support substrate 500 is a direction perpendicular to the fifth principal surface 500a. The thickness direction DR3 of the support substrate 500 is the same as the thickness direction Dr1 of the first layer 100. The first electrode layer 710, the second electrode layer 720, and the light emitting layer 730 are thin films. For example, the first electrode layer 710 is configured with a transparent material having conductivity. The transparent material constituting the first electrode layer 710 includes at least one of indium tin oxide (ITO), indium zinc oxide (IZO), zinc oxide (ZnO), and IGZO. The first electrode layer 710 is in contact with the sixth principal surface 500b. The second electrode layer 720 is configured with a conductive material. For example, the conductive material constituting the second electrode layer 720 includes copper (Cu), aluminum (Al), tungsten (W), gold (Au), and silver (Ag). For example, the light emitting layer 730 is configured with an inorganic light emitting device having a semiconductor stack layer. The light emitting layer 730 may include an organic light emitting material. For example, the first electrode layer 710, the second electrode layer 720, and the light emitting layer 730 are collectively formed through a thin film stacking process.

When different voltages are applied to the first electrode layer 710 and the second electrode layer 720, the light emitting layer 730 emits light. The light emitted from the light emitting layer 730 passes through the first electrode layer 710 and is incident on the support substrate 500.

The configuration shown in FIG. 12, other than the above, is similar to the configuration shown in FIG. 4.

The solid-state imaging device 17 need not include at least one of the wirings 230, the plurality of transistors 240, and the vias 250. A plurality of first microlenses 300 may be similar to the plurality of first microlenses 300 in the solid-state imaging device 12 shown in FIG. 5. A plurality of second microlenses 310 may be similar to the plurality of second microlenses 310 in the solid-state imaging device 12 shown in FIG. 5. The first layer 100 may be changed to the first layer 101 in the solid-state imaging device 13 shown in FIG. 6. The solid-state imaging device 17 may include the filter layer 600 in the solid-state imaging device 14 shown in FIG. 7. The solid-state imaging device 17 may include the filter 620 in the solid-state imaging device 15 shown in FIG. 10 or the solid-state imaging device 16 shown in FIG. 11.

The solid-state imaging device 17 according to the seventh embodiment can reduce light which is not irradiated to the object 900 and is incident on the photoelectric conversion elements 110.

Since the light emitting element 700 is arranged, the solid-state imaging device 17 is miniaturized as compared with a device in which a light source and a solid-state imaging device are separated from each other.

### Modification Example of Seventh Embodiment

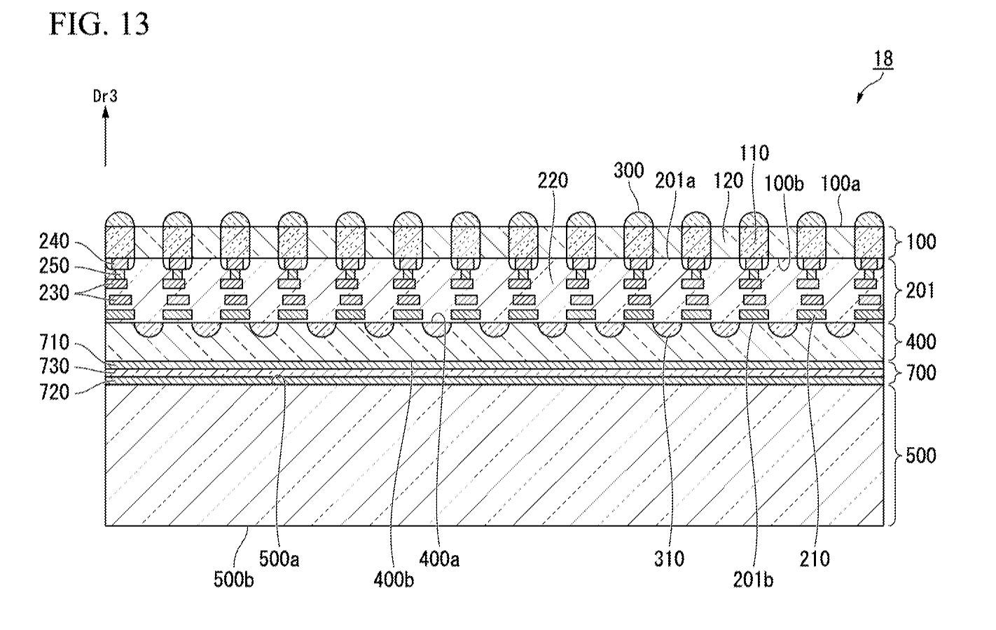

FIG. 13 shows a configuration of a solid-state imaging device 18 according to a modification example of the seventh embodiment of the present invention. FIG. 13 shows a section of the solid-state imaging device 18. The configuration shown in FIG. 13, which is different from that shown in FIG. 12, will be described.

The solid-state imaging device 18 includes the third layer 400, the support substrate 500, and the light emitting element 700. The third layer 400 includes a plurality of second microlenses 310. The third layer 400 faces the fourth principal surface 201b. The third layer 400 is arranged between the second layer 201 and the light emitting element 700. The third layer 400 allows light incident on the third layer 400 to pass therethrough. The support substrate 500 includes the fifth principal surface 500a and the sixth principal surface 500b. The fifth principal surface 500a and the sixth principal surface 500b face opposite directions. The light emitting element 700 includes the first electrode layer 710, the second electrode layer 720, and the light emitting layer 730. The first electrode layer 710, the second electrode layer 720, and the light emitting layer 730 are stacked in the thickness direction DR3 of the support substrate 500. The first electrode layer 710 faces the third layer 400. The second electrode layer 720 faces the fifth principal surface 500a. The light emitting layer 730 is arranged between the first electrode layer 710 and the second electrode layer 720.

The first electrode layer 710 faces the eighth principal surface 400b and is in contact with the eighth principal surface 400b. The second electrode layer 720 is in contact with the fifth principal surface 500a. When different voltages are applied to the first electrode layer 710 and the second electrode layer 720, the light emitting layer 730 emits light. The light emitted from the light emitting layer 730 passes through the first electrode layer 710 and is incident on the third layer 400.

The support substrate 500 need not allow light to pass therethrough. Therefore, a material constituting the support substrate 500 need not be a transparent material.

The configuration shown in FIG. 13, other than the above, is similar to the configuration shown in FIG. 12.

The solid-state imaging device 18 need not include at least one of the wirings 230, the plurality of transistors 240, and the vias 250. A plurality of first microlenses 300 may be similar to the plurality of first microlenses 300 in the solid-state imaging device 12 shown in FIG. 5. A plurality of second microlenses 310 may be similar to the plurality of second microlenses 310 in the solid-state imaging device 12 shown in FIG. 5. The first layer 100 may be changed to the first layer 101 in the solid-state imaging device 13 shown in FIG. 6. The solid-state imaging device 18 may include the filter layer 600 in the solid-state imaging device 14 shown in FIG. 7. The solid-state imaging device 18 may include the filter 620 in the solid-state imaging device 15 shown in FIG. 10 or in the solid-state imaging device 16 shown in FIG. 11. When the filter 620 is arranged between the third layer 400 and the support substrate 500, the filter 620 is arranged between the third layer 400 and the light emitting element 700.

While preferred embodiments of the invention have been described and shown above, it should be understood that these are exemplars of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A solid-state imaging device, comprising:
a first layer which includes a first principal surface, a second principal surface, a plurality of photoelectric conversion elements, and a first light transmission layer, the first principal surface and the second principal surface facing opposite directions, the plurality of photoelectric conversion elements being configured to receive light incident on the first principal surface, and the first light transmission layer allowing light, which is incident on a second region different from a first region corresponding to the plurality of photoelectric conversion elements in the second principal surface, to be emitted from the first principal surface;
a second layer which includes a third principal surface, a fourth principal surface, a first light blocking film, and a second light transmission layer, the third principal surface and the fourth principal surface facing opposite directions, the third principal surface facing the second principal surface, the first light blocking film being arranged in a third region of the second layer corresponding to the plurality of photoelectric conversion elements and blocking light incident on the fourth principal surface, and the second light transmission layer allowing light, which is incident on a fifth region of the fourth principal surface that is different from a fourth region of the fourth principal surface corresponding to the first light blocking film and that corresponds to the first light transmission layer, to be emitted from the third principal surface;
a plurality of first microlenses which are arranged in a sixth region of the first principal surface which corresponds to the plurality of photoelectric conversion elements and have a convex shape toward an outer side of the first principal surface; and
a plurality of second microlenses which are arranged in the fifth region and have a convex shape toward an outer side of the fourth principal surface.

2. The solid-state imaging device according to claim 1, further comprising:
a support substrate which includes a fifth principal surface and a sixth principal surface and allows light incident on the support substrate to pass therethrough; and
a third layer which includes the plurality of second microlenses, faces the fourth principal surface, is arranged between the second layer and the support substrate, and allows light incident on the third layer to pass therethrough,
wherein the fifth principal surface and the sixth principal surface face opposite directions and the fifth principal surface faces the third layer.

3. The solid-state imaging device according to claim 1, wherein the plurality of first microlenses are arranged only in the sixth region, and
the plurality of second microlenses are arranged only in the fifth region.

4. The solid-state imaging device according to claim 1, wherein the plurality of first microlenses are arranged in the sixth region and a seventh region of the first principal surface which corresponds to the first light transmission layer.

5. The solid-state imaging device according to claim 1, wherein the plurality of second microlenses are arranged in the fifth region and the fourth region.

6. The solid-state imaging device according to claim 1, further comprising:
a plurality of transistors which are electrically connected to the plurality of photoelectric conversion elements and are arranged between the plurality of photoelectric conversion elements and the fourth principal surface.

7. The solid-state imaging device according to claim 1, wherein the second layer further comprises a plurality of wirings including a metal.

8. The solid-state imaging device according to claim 1, wherein a focal distance of each of the plurality of first microlenses is shorter than a focal distance of each of the plurality of second microlenses.

9. The solid-state imaging device according to claim 1, wherein a width of the first light transmission layer arranged between two adjacent photoelectric conversion elements is larger than an interval between the first light blocking films respectively corresponding to the two adjacent photoelectric conversion elements.

10. The solid-state imaging device according to claim 1, wherein the first light transmission layer is arranged in a groove formed between the plurality of photoelectric conversion elements.

11. The solid-state imaging device according to claim 10, wherein the first layer further comprises:
a second light blocking film which is arranged on a side surface of the groove and is configured to block light incident on the first light transmission layer.

12. The solid-state imaging device according to claim 1, further comprising:
a plurality of color filters which are arranged in the sixth region and are arranged between the plurality of photoelectric conversion elements and the plurality of first microlenses.

13. The solid-state imaging device according to claim 1, further comprising:
a filter which has a structure in which a plurality of films including a dielectric are stacked; and
a third layer which includes the plurality of second microlenses, faces the fourth principal surface, is arranged between the second layer and the filter, and allows light incident on the third layer to pass therethrough.

14. The solid-state imaging device according to claim 1, further comprising:
a filter which has a structure in which a plurality of films including a dielectric are stacked and is arranged between the second layer and the plurality of second microlenses.

15. The solid-state imaging device according to claim 1, wherein the first light blocking film includes a conductive material and a power supply voltage or a ground voltage is applied to the first light blocking film.

16. The solid-state imaging device according to claim 2, further comprising:
a light emitting element which includes a first electrode layer, a second electrode layer, and a light emitting layer,
wherein the first electrode layer, the second electrode layer, and the light emitting layer are stacked in a thickness direction of the support substrate, the first electrode layer faces the sixth principal surface, and the light emitting layer is arranged between the first electrode layer and the second electrode layer.

17. The solid-state imaging device according to claim 1, further comprising:
a third layer which includes the plurality of second microlenses, faces the fourth principal surface, is arranged between the second layer and a light emitting element, and allows light incident on the third layer to pass therethrough;
a support substrate which includes a fifth principal surface and a sixth principal surface, the fifth principal surface and the sixth principal surface facing opposite directions; and
the light emitting element which includes a first electrode layer, a second electrode layer, and a light emitting layer,
wherein the first electrode layer, the second electrode layer, and the light emitting layer are stacked in a thickness direction of the support substrate, the first electrode layer faces the third layer, the second electrode layer faces the fifth principal surface, and the light emitting layer is arranged between the first electrode layer and the second electrode layer.

18. The solid-state imaging device according to claim 16, wherein the light emitting layer includes an organic light emitting material.

19. The solid-state imaging device according to claim 17, wherein the light emitting layer includes an organic light emitting material.
